(19) United States
(12) Patent Application Publication
(10) Pub. No.: US 2023/0110785 A1
(43) Pub. Date: Apr. 13, 2023

(54) POLYCRYSTALLINE DIAMOND SAMPLING VALVE

(71) Applicant: Sensia LLC, Houston, TX (US)

(72) Inventor: Gary Potten, Spring, TX (US)

(21) Appl. No.: 17/963,337

(22) Filed: Oct. 11, 2022

Related U.S. Application Data
(63) Continuation of application No. PCT/US22/46127, filed on Oct. 7, 2022.
(60) Provisional application No. 63/253,837, filed on Oct. 8, 2021.
(60) Provisional application No. 63/253,837, filed on Oct. 8, 2021.

(51) Int. Cl. G01N 1/20

(52) U.S. Cl. CPC G01N 1/2035

(57) ABSTRACT

A relief valve in a hydrocarbon site is shown. The relief valve is disposed within a housing. The housing includes a first engagement component coupled with a valve spring and a second engagement component axially aligned with the first engagement component. The second engagement component is configured to engage with the first engagement component during an operation cycle to translate the stem along the axis in response to the pressure within the relief valve being above the predetermined threshold. In some embodiments, the second engagement component is formed at least in part of a carbide substrate.

### CROSS-REFERENCE TO RELATED CASES

This U.S. Patent Application is a continuation of International Patent Application No. PCT/US2022/046127, filed Oct. 7, 2022, which claims priority to U.S. Provisional Application No. 63/253,837, filed Oct. 8, 2021. This application also claims priority to U.S. Provisional Application No. 63/253,837, filed Oct. 8, 2021. The contents of each of these applications are incorporated herein by reference in their entireties for all purposes.

### BACKGROUND

The present disclosure relates to hydrocarbon systems. More specifically, the present disclosure relates to sampling valves in hydrocarbon systems.

A hydrocarbon system may include one or more pipelines (e.g., tank lines, conduits, pipes, etc.) configured to facilitate fluid from one location to another. The fluid may include crude oil, hydrocarbon residue, refined distillates, and any other refined product produced in a hydrocarbon system. In some embodiments, the hydrocarbon system will incorporate one or more sampling relief valves to maintain back pressure in the system and to allow a sample taken from one or more of the pipelines and provide the sample to an analytics system (e.g., a sample receiver, an online analyzer, etc.).

The sampling relief valve may open frequently (e.g., hundreds of times a day, thousands of times a day, etc.) and can be subject to significant erosion, wear and therefore premature failure. Additionally, sediment in the crude oil or hydrocarbon product may compound the degradation of the valve. The steam and seat of the sampling valve may be manufactured from stainless steel, and failure of the sample valve may result in a sample receiver overfilling, leakage of the fluid before, during, or after a sample is taken, and increased safety issues.

### SUMMARY

This summary is illustrative only and is not intended to be in any way limiting. Other aspects, inventive features, and advantages of the devices or processes described herein will become apparent in the detailed description set forth herein, taken in conjunction with the accompanying figures, wherein like reference numerals refer to like elements.

One implementation of the present disclosure is a relief valve. The relief valve includes a first engagement component, a second engagement component, and a carbide substrate. The first engagement component is coupled with a valve spring. The second engagement component is axially aligned with the first engagement component and configured to engage with the first engagement component during an operation cycle to translate the first engagement component along an axis in response to a pressure of a fluid within the relief valve being above a predetermined threshold. The carbide substrate is disposed between the first engagement component and the second engagement component when the second engagement component engages the first engagement component.

In some embodiments, the carbide substrate is fused with diamond particles to form polycrystalline diamond (PCD).

In some embodiments, the operation cycle includes engaging, in response to an increase in the pressure, a top surface of the second engagement component with a bottom surface of the first engagement component; permitting a fluid path from a pipeline to an outlet of the relief valve; disengaging, in response to a decrease in the pressure, the top surface of the second engagement component with the bottom surface of the first engagement component; and restricting the fluid path from the pipeline to the outlet of the relief valve.

In some embodiments, the first engagement component is a valve stem of the relief valve, the second engagement component is a valve seat of the relief valve, the pipeline is an unrefined oil pipeline configured to provide the fluid to a refining process, and the fluid is crude oil.

In some embodiments, the operation cycle of the relief valve is performed more than one thousand times per day.

In some embodiments, the relief valve is a sampling relief valve configured to obtain a sample of the fluid flowing through a pipeline.

In some embodiments, the relief valve is fluidly coupled with an analytics system. The analytics system is configured to receive the sample from the relief valve in response to a completion of the operation cycle and analyze one or more properties of the sample to determine an amount of sediment within the sample.

In some embodiments, at least one of the first engagement component or the second engagement component is manufactured from a Tungsten Cobalt alloy of between 5 percent and 40 percent Cobalt with respect to Tungsten by atomic weight.

Another implementation of the present disclosure is a relief valve in a hydrocarbon site. The relief valve includes an inlet port, a first engagement component, a second engagement component, and a carbide substrate. The inlet port is configured to receive a fluid from a pipeline. The second engagement component is configured to engage with the first engagement component during an operation cycle such that the second engagement component causes the first engagement component to translate the stem along an axis in response to a pressure of the fluid within the relief valve being above a predetermined threshold. The carbide substrate is disposed between the first engagement component and the second engagement component.

In some embodiments, the carbide substrate is fused with diamond particles to form polycrystalline diamond (PCD).

In some embodiments, the operation cycle includes engaging, in response to an increase in the pressure, a top surface of the second engagement component with a bottom surface of the first engagement component; permitting a fluid path from the pipeline to an outlet of the relief valve; disengaging, in response to the decrease in the pressure, the top surface of the second engagement component with the bottom surface of the first engagement component; and restricting the fluid path from the pipeline to the outlet of the relief valve.

In some embodiments, the first engagement component is a valve stem of the relief valve, the second engagement component is a valve seat of the relief valve, the pipeline is an unrefined oil pipeline configured to provide the fluid to a refining process, and the fluid is crude oil.

In some embodiments, the operation cycle of the relief valve is performed more than one thousand times per day.

In some embodiments, the relief valve is a sampling relief valve configured to obtain a sample of the fluid flowing through the pipeline.

In some embodiments, the relief valve is fluidly coupled with an analytics system. The analytics system is configured to receive the sample from the relief valve in response to a completion of the operation cycle, and analyze one or more properties of the sample to determine an amount of sediment within the sample.

Another implementation of the present disclosure is a valve including a first engagement component, a second engagement component configured to engage with the first engagement component, and a carbide substrate disposed between the first engagement component and the second engagement component such that the carbide substrate contacts the first engagement component and the second engagement component when the second engagement component engages the first engagement component.

In some embodiments, the carbide substrate is fused with diamond particles to form polycrystalline diamond (PCD).

In some embodiments, the second engagement component is configured to engage with the first engagement component during an operation cycle. The operation cycle includes engaging, in response to an increase in a pressure of a fluid within the valve, a top surface of the second engagement component with a bottom surface of the first engagement component, permitting a fluid path from an inlet of the valve to an outlet of the valve, disengaging, in response to a decrease in the pressure of the fluid within the valve, the top surface of the second engagement component with the bottom surface of the first engagement component, and restricting the fluid path from the inlet of the valve to the outlet of the relief valve.

In some embodiments, the first engagement component is a valve stem of the relief valve, the second engagement component is a valve seat of the relief valve, the inlet is configured to receive the fluid from an unrefined oil pipeline configured to provide the fluid to a refining process, and the fluid is crude oil.

In some embodiments, the valve is fluidly coupled with an analytics system. The analytics system is configured to: receive the fluid from the relief valve in response to a completion of the operation cycle, and analyze one or more properties of the fluid to determine an amount of sediment within the fluid.

### BRIEF DESCRIPTION OF THE DRAWINGS

Certain embodiments of the disclosure will hereafter be described with reference to the accompanying drawings, wherein like reference numerals denote like elements. It should be understood, however, that the accompanying figures illustrate the various implementations described herein and are not meant to limit the scope of various technologies described herein.

FIG. 1 is a perspective view of a hydrocarbon site equipped with well devices, according to some embodiments.

FIG. 2 is a block diagram of a hydrocarbon system, which can be performed at least in part within the hydrocarbon site of FIG. 1, according to some embodiments.

FIG. 3A is a cross-sectional diagram of a relief valve, which can be implemented in the hydrocarbon site of FIG. 2, according to some embodiments.

FIG. 3B is a cross-sectional diagram of the seat/stem assembly of the relief valve of FIG. 3A, according to some embodiments.

FIG. 3C is a cross-sectional diagram of the seat/stem assembly of the relief valve of FIG. 3A, according to some embodiments.

FIG. 4 is a block diagram of a process for permitting fluid flow through a relief valve, which can be performed by the sampling relief valve of FIG. 3A, according to some embodiments.

### DETAILED DESCRIPTION

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another.

Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present invention, the articles “a,” “an,” “the,” and “said” are intended to mean that there are one or more of the elements. The terms “comprising,” “including,” and “having” are intended to be inclusive and mean that there may be additional elements other than the listed element.

### Overview

Referring generally to the FIGURES, systems and methods for a sampling valve (e.g., sampling relief valve, etc.) with one or more interior components (e.g., the valve stem, the valve seat, etc.) at least partially manufactured/coated with a substrate (e.g., polycrystalline diamond, etc.) to improve durability. This may allow the sampling relief valve to maintain durability and/or operability as the valve is subject to frequent (e.g., hundreds of times per day, thousands of times per day, etc.) actuation and/or facilitates fluid flow of a fluid that can cause excessive wear on valve systems, such as crude oil that contains various sediments. As such, a sampling relief valve at least partially manufactured/coated with a substrate, as described herein, can significantly extend the life of the valve.

While the systems and methods disclosed herein generally refer to valve components equipped with polycrystalline diamond (PCD) material (e.g., manufactured out of PCD, coated with PCD, etc.), other durable materials can also be considered. For example, the engagement components of a sampling valve (e.g., the valve and stem, etc.) can be manufactured at least in part out of carbine, high-content cobalt, high-content cobalt bonded with tungsten, or any combination thereof. For example, the engagement components of the sampling valve may be manufactured out of tungsten and alloys thereof In some embodiments, the engagement components are manufactured from a Tungsten Cobalt alloy of between 5 percent and 40 percent Cobalt with respect to Tungsten by atomic weight (e.g., 20 percent Cobalt and 80 percent Tungsten by atomic weight). In some embodiments, the alloy includes tertiary metals such as titanium, tantalum, etc. In some embodiments, the material for the engagement component is more malleable than pure Tungsten to prevent cracking due to the interface with the polycrystalline diamond (PCD) material. In some embodiments, the engagement components may be manufactured from other materials. Similarly, while the systems and methods disclosed herein are generally referring to the stem and seat of valves as being manufactured/coated with PCD, any components that engage, disengage during an operation cycle of the valve 0 can be considered.

### Flow System Overview

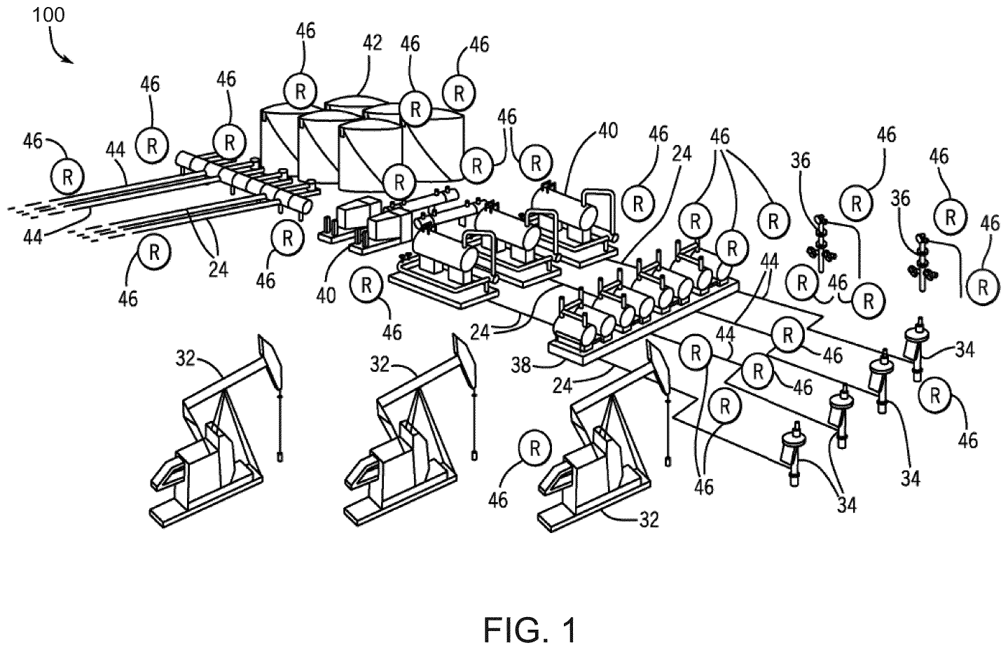

Referring now to FIG. 1, a hydrocarbon site 100 may be an area in which hydrocarbons, such as crude oil and natural gas, may be extracted from the ground, processed, and stored. As such, the hydrocarbon site 100 may include a number of wells and a number of well devices that may control the flow of hydrocarbons being extracted from the wells. In one embodiment, the well devices at the hydrocarbon site 100 may include any device equipped to monitor and/or control production of hydrocarbons at a well site. As such, the well devices may include pumpjacks 32, submersible pumps 34, well trees 36, and other devices for assisting the monitoring and flow of liquids or gasses, such as petroleum, natural gasses and other substances. After the hydrocarbons are extracted from the surface via the well devices, the extracted hydrocarbons may be distributed to other devices such as wellhead distribution manifolds 38, separators 40, storage tanks 42, and other devices for assisting the measuring, monitoring, separating, storage, and flow of liquids or gasses, such as petroleum, natural gasses and other substances. At the hydrocarbon site 100, the pumpjacks 32, submersible pumps 34, well trees 36, wellhead distribution manifolds 38, separators 40, and storage tanks 42 may be connected together via a network of pipelines 44. As such, hydrocarbons extracted from a reservoir may be transported to various locations at the hydrocarbon site 100 via the network of pipelines 44.

The pumpjack 32 may mechanically lift hydrocarbons (e.g., oil) out of a well when a bottom hole pressure of the well is not sufficient to extract the hydrocarbons to the surface. The submersible pump 34 may be an assembly that may be submerged in a hydrocarbon liquid that may be pumped. As such, the submersible pump 34 may include a hermetically sealed motor, such that liquids may not penetrate the seal into the motor. Further, the hermetically sealed motor may push hydrocarbons from underground areas or the reservoir to the surface.

The well trees 36 or Christmas trees may be an assembly of valves, spools, and fittings used for natural flowing wells. As such, the well trees 36 may be used for an oil well, gas well, water injection well, water disposal well, gas injection well, condensate well, and the like. The wellhead distribution manifolds 38 may collect the hydrocarbons that may have been extracted by the pumpjacks 32, the submersible pumps 34, and the well trees 36, such that the collected hydrocarbons may be routed to various hydrocarbon processing or storage areas in the hydrocarbon site 100.

The separator 40 may include a pressure vessel that may separate well fluids produced from oil and gas wells into separate gas and liquid components. For example, the separator 40 may separate hydrocarbons extracted by the pumpjacks 32, the submersible pumps 34, or the well trees 36 into oil components, gas components, and water components. After the hydrocarbons have been separated, each separated component may be stored in a particular storage tank 42. The hydrocarbons stored in the storage tanks 42 may be transported via the pipelines 44 to transport vehicles, refineries, and the like.

The well devices may also include monitoring systems that may be placed at various locations in the hydrocarbon site 100 to monitor or provide information related to certain aspects of the hydrocarbon site 100. As such, the monitoring system may be a controller, a remote terminal unit (RTU), or any computing device that may include communication abilities, processing abilities, and the like. For discussion purposes, the monitoring system will be embodied as the RTU 46 throughout the present disclosure. However, it should be understood that the RTU 46 may be any component capable of monitoring and/or controlling various components at the hydrocarbon site 100. The RTU 46 may include sensors or may be coupled to various sensors that may monitor various properties associated with a component at the hydrocarbon site 10.

The RTU 46 may then analyze the various properties associated with the component and may control various operational parameters of the component. For example, the RTU 46 may measure a pressure or a differential pressure of a well or a component (e.g., storage tank 42) in the hydrocarbon site 100. The RTU 46 may also measure a temperature of contents stored inside a component in the hydrocarbon site 100, an amount of hydrocarbons being processed or extracted by components in the hydrocarbon site 100, and the like. The RTU 46 may also measure a level or amount of hydrocarbons stored in a component, such as the storage tank 42. In certain embodiments, the RTU 46 may be iSens-GP Pressure Transmitter, iSens-DP Differential Pressure Transmitter, iSens-MV Multivariable Transmitter, iSens-T2 Temperature Transmitter, iSens-L Level Transmitter, or Isens-1O Flexible 1/0 Transmitter manufactured by vMonitor® of Houston, Tex. In some embodiments, hundreds or even thousands of different devices (e.g., including internal products and 3^{rd} party products) may be supported and the previously mentioned devices are intended as only exemplary embodiments and are not intended to be limiting.

In one embodiment, the RTU 46 may include a sensor that may measure pressure, temperature, fill level, flow rates, and the like. The RTU 46 may also include a transmitter, such as a radio wave transmitter, that may transmit data acquired by the sensor via an antenna or the like. The sensor in the RTU 46 may be wireless sensors that may be capable of receive and sending data signals between RTUs 26. To power the sensors and the transmitters, the RTU 46 may include a battery or may be coupled to a continuous power supply. Since the RTU 46 may be installed in harsh outdoor and/or explosion-hazardous environments, the RTU 46 may be enclosed in an explosion-proof container that may meet certain standards established by the National Electrical Manufacturer Association (NEMA) and the like, such as a NEMA 4X container, a NEMA 7X container, and the like.

The RTU 46 may transmit data acquired by the sensor or data processed by a processor to other monitoring systems, a router device, a supervisory control and data acquisition (SCADA) device, or the like. As such, the RTU 46 may enable users to monitor various properties of various components in the hydrocarbon site 100 without being physically located near the corresponding components. The RTU 46 can be configured to communicate with the devices at the hydrocarbon site 100 as well as mobile computing devices via various networking protocols.

In operation, the RTU 46 may receive real-time or near real-time data associated with a well device. The data may include, for example, tubing head pressure, tubing head temperature, case head pressure, flowline pressure, wellhead pressure, wellhead temperature (e.g., temperature measurements from downhole (in the well), etc.), and the like. In any case, the RTU 46 may analyze the real-time data with respect to static data that may be stored in a memory of the RTU 46. The static data may include a well depth, a tubing length, a tubing size, a choke size, a reservoir pressure, a bottom hole temperature, well test data, fluid properties of the hydrocarbons being extracted, and the like. The RTU 46 may also analyze the real-time data with respect to other data acquired by various types of instruments (e.g., water cut meter, multiphase meter) to determine an inflow performance relationship (IPR) curve, a desired operating point for the wellhead 30, key performance indicators (KPis) associated with the wellhead 30, wellhead performance summary reports, and the like. Although the RTU 46 may be capable of performing the above-referenced analyses, the RTU 46 may not be capable of performing the analyses in a timely manner. Moreover, by just relying on the processor capabilities of the RTU 46, the RTU 46 is limited in the amount and types of analyses that it may perform. Moreover, since the RTU 46 may be limited in size, the data storage abilities may also be limited. The RTU 46 can be configured to receive time series data and provide time-series data.

In certain embodiments, the RTU 46 may establish a communication link with the cloud-based computing system 12 described above. As such, the cloud-based computing system 12 may use its larger processing capabilities to analyze data acquired by multiple RTUs 26. Moreover, the cloud-based computing system 12 may access historical data associated with the respective RTU 46, data associated with well devices associated with the respective RTU 46, data associated with the hydrocarbon site 100 associated with the respective RTU 46 and the like to further analyze the data acquired by the RTU 46. The cloud-based computing system 12 is in communication with the RTU via one or more servers or networks (e.g., the Internet, a private corporate network, etc.).

### Hydrocarbon System

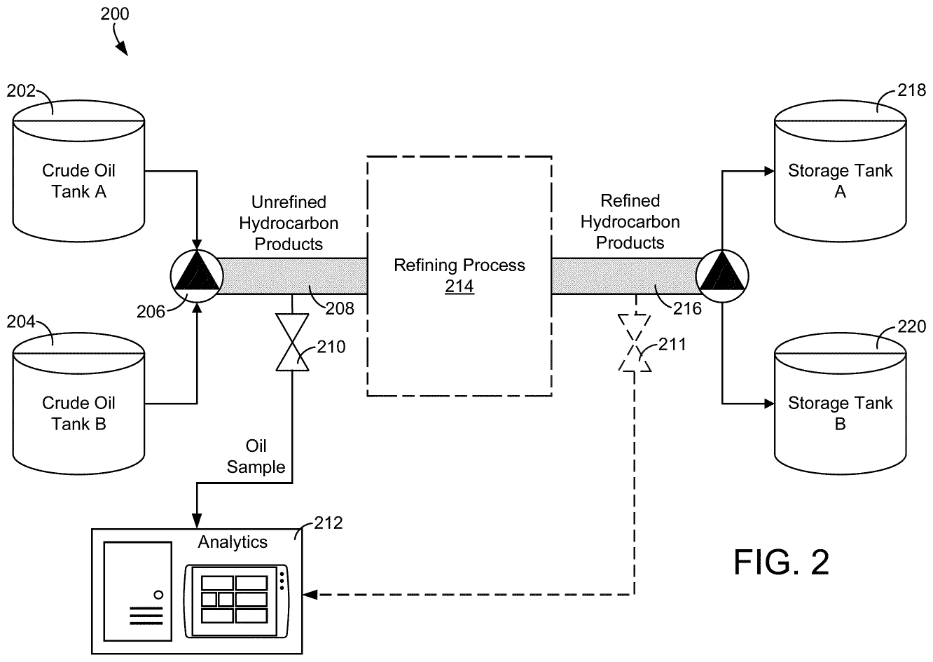

Referring now to FIG. 2, a system 200 for processing crude oil into refined hydrocarbon products is shown, according to some embodiments. System 200 may be performed partially or entirely at hydrocarbon site 100, as described above. System 200 is shown to include crude oil tanks 202, 204, pump 206, unrefined hydrocarbon products pipeline (“pipeline”) 208, valves 210, 211, analytics processing circuity (“analytics”) 212, refining process 214, refined hydrocarbon products pipeline (“pipeline”) 216, and storage tanks 218, 220.

In a general embodiment, system 200 obtains crude oil and other unrefined hydrocarbon products from tanks 202-204 and pumps the unrefined hydrocarbon products into pipeline 208 to be processed (e.g., within an atmospheric distillation unit, in a fractionator, in a fluid catalytic cracking unit, etc.). Upon being refined, the hydrocarbon products may be pumped in storage tanks 218, 220 for distribution. During this process, one or more valves (e.g., valve 210, valve 211) coupled to the pipeline 208, may “sample” the fluid flowing through pipelines 208, 216 for analytic purposes. As used herein “sample” or “sampling” a fluid, in addition to the plain meaning of the words, refers to removing at least a portion of the fluid by one or more valves (e.g., valve 210, valve 211). For example, as shown in FIG. 2, valve 210 acts as a sampling relief valve to sample the unrefined hydrocarbon products being piped to refining process 214. Valve 210 may then facilitate flow of the received fluid to analytics 212 for processing.

Of course, the reining process for crude oil may be complex and involve many stages; thus, the pipelines shown and the stages the pipelines are at are merely meant to be exemplary and should not be considered limiting. Any number of fluid pipelines can be fluidly coupled with sampling valves (e.g., a pipeline facilitating atmospheric residuum from an atmospheric distillation unit to a vacuum distillation unit, a pipeline facilitating residual oil from a vacuum distillation unit to a coker, etc.). Additionally, any number of sampling valves and/or sampling relief valves can be fluidly coupled to the pipelines 208, 216 shown in FIG. 2 or any other pipeline typically configured to facilitate unrefined, partially refined, or refined hydrocarbon products within refining process 214.

Storage tanks 202, 204 may represent the mechanical components and/or methods for storing and/or providing crude oil into system 200. As disclosed herein, the terms “petroleum” and “crude oil” may be used interchangeably when referring to the mixture of hydrocarbons received prior to oil refining. In some embodiments, the oil stored in storage tanks 202, 204 has an American Petroleum Institute (API) gravity of 15-45 degrees, wherein a high API indicates a lower density crude oil and a low API indicates a higher density crude oil. In some embodiments, the oil stored in storage tanks 202, 204 has a lower or higher API gravity. In some embodiments, the level of concarbon content (CCR) (e.g., Conradson carbon residue, etc.) is measured to provide an indication of the coke-forming tendencies of the crude oil, prior to providing crude oil to system 100 via oil tanks 102-108. The crude oil stored in storage tanks 202, 204 may be recovered through various forms of oil drilling and/or natural petroleum springs. A pumping system (e.g., pump 206, etc.) may then transfer the received crude oil to store in storage tanks 202, 204 (not shown) and provide the crude oil to further processing. Similarly, storage tanks 218, 220 may represent the mechanical components and/or methods for storing and/or receiving the refined hydrocarbon products from refining process 214.

Refining process 214 may generally be configured to transform crude oil (or other crude petroleum products) into more useful products (e.g., gasoline, petrol, kerosene, jet fuel, etc.). FIG. 2 depicts one example of system 200 for refining crude oil, but it should be understood that the systems and methods described herein are not limited to any particular configuration of system 200. For example, other embodiments of system 200 may include refinery tools (e.g., a de-salter for the crude oil, hydrocrackers, hydrotreaters, etc.), different arrangements or configurations of system 200 that include the same components, more or fewer storage tanks and/or storage containers, and other modifications to system 200.

It is further noted that although system 200 is described primarily as refining crude oil, it should be understood that the systems and methods described herein can be used to refine or produce any of a variety of petroleum products. For example, system 200 can be operated to produce butane, methane, diesel fuel, fuel oil, gasoline, kerosene, liquefied natural gas, liquefied petroleum gas, propane, microcrystalline wax, napalm, naphtha, naphthalene, paraffin wax, petroleum jelly, petroleum wax, refined asphalt, refined bitumen, refined petroleum gas, slack wax, sulfur, petroleum coke, petrochemicals, or any other type of petroleum product. In general, system 200 may be configured to convert one or more input petroleum products into one or more output or derived petroleum products.

Analytics 212 may be configured to receive samples of the hydrocarbon products within system 200 and analyze the samples for safety, quality control, analytic, and/or other testing purposes. While not shown, analytics 212 may be or include one or more processing devices (e.g., a processor, ASIC, FPGA, etc.) and may be embedded in one or more devices (e.g., field controller, supervisory controller, PDA, laptop, tablet, smartphone application, etc.).

Analytics 212 may include a communications interface and a processing circuit that includes one or more processors and memory. The processing circuit can be communicably connected to the communications interface such that the processing circuit and the various components thereof can send and receive data via the communications interface. The processor can be implemented as a general purpose processor, an application specific integrated circuit (ASIC), one or more field programmable gate arrays (FPGAs), a group of processing components, or other suitable electronic processing components.

The communications interface can be or include wired or wireless communications interfaces (e.g., jacks, antennas, transmitters, receivers, transceivers, wire terminals, etc.) for conducting data communications. In various embodiments, communications via the communications interface can be direct (e.g., local wired or wireless communications) or via a communications network (e.g., a WAN, the Internet, a cellular network, etc.). For example, the communications interface can include an Ethernet card and port for sending and receiving data via an Ethernet-based communications link or network. In another example, the communications interface can include a Wi-Fi transceiver for communicating via a wireless communications network. In another example, the communications interface can include cellular or mobile phone communications transceivers.

The memory (e.g., memory, memory unit, storage device, etc.) can include one or more devices (e.g., RAM, ROM, Flash memory, hard disk storage, etc.) for storing data and/or computer code for completing or facilitating the various processes, layers and modules described in the present disclosure. The memory can be or include volatile memory or non-volatile memory. The memory can include database components, object code components, script components, or any other type of information structure for supporting the various activities and information structures described in the present disclosure. According to an example embodiment, the memory is communicably connected to the processor via the processing circuit and includes computer code for executing (e.g., by the processing circuit and/or the processor) one or more processes described herein. In some embodiments, analytics 212 is implemented within a single computer (e.g., one server, one housing, etc.). In various other embodiments analytics 212 can be distributed across multiple servers or computers (e.g., that can exist in distributed locations).

Analytics 212 may be configured to receive oil samples (e.g., crude oil, refined hydrocarbon product samples, etc.) and perform analyses on one or more properties of the oil samples. For example, a processing component of analytics 212 (e.g., a controller, an FPGA, an ADIC, etc.) determines the amount of sediment within the sample, and provides the information to a user (e.g., via an application, via a display, etc.).

### Sampling Relief Valve

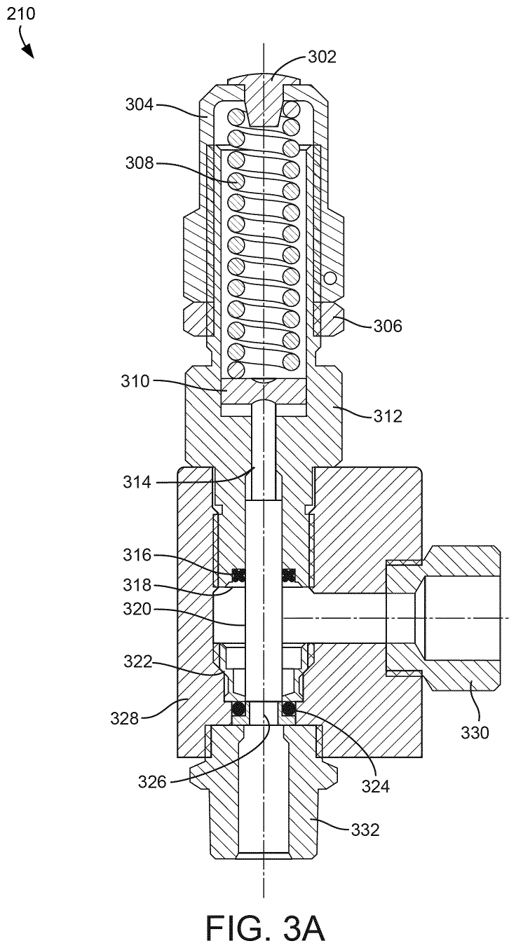

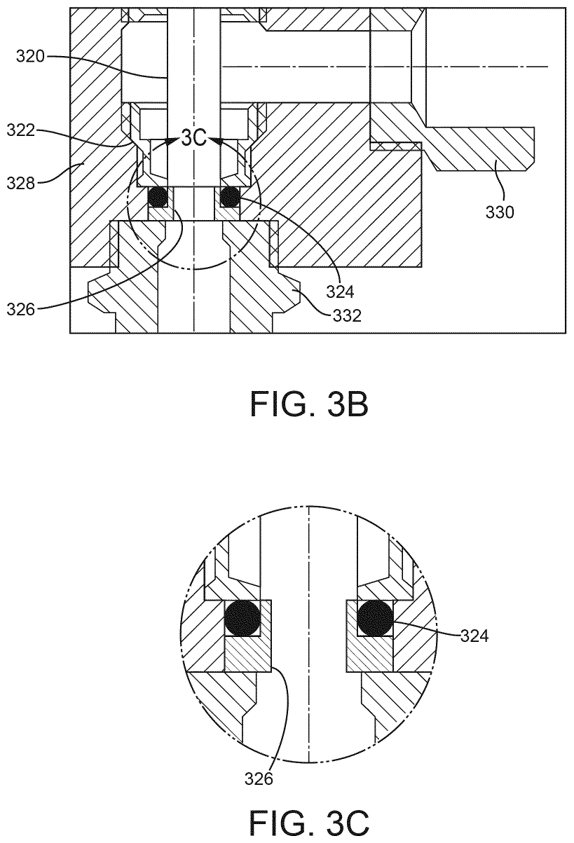

Referring generally to FIGS. 3A-C, several cross-sectional diagrams of different portions of valve 210 are shown, according to some embodiments. Referring specifically to FIG. 3A, a cross-sectional diagram of a valve 210 is shown, according to some embodiments. Valve 210 may act as a sampling relief valve, a pressure relief valve, a sampling valve, or any combination thereof. Additionally, while the systems and methods disclosed herein generally refer to a sampling relief valve based on spring motion, and type of actuation to provide sampling and/or relief may be implemented. For example, valve 210 may be coupled with an actuator with wireless communication capabilities such that the actuator can receive control signals and actuate valve 210 accordingly. While FIGS. 3A-C generally disclose a spring-based relief mechanism, other types of mechanisms are considered and are described in detail below.

In some embodiments, valve 210 may be configured to provide a pressure relief if the pressure within pipeline 208 or one or more of the other pipelines in system 200 are at an abnormally high pressure. In another example, valve 210 may be configured to open and allow fluid within pipeline 208 as a sample (e.g., open and close substantially quickly to allow only a portion of the fluid to exit pipeline 208 for sampling purposes, etc.).

In such an embodiment, valve 210 may be actuated automatically (e.g., based on a timer, based on a measured flow reading, based on a measured pressure reading, etc.). For example, a pressure sensor is communicably connected to valve 210 (e.g., wired to valve 210, via a smart actuator (not shown), etc.), such that when a pressure value is obtained that is higher than a predetermined threshold, valve 210 may be actuated to open and provide pressure relief to system 200. In other embodiments, valve 210 may be actuated based on manual control. For example, a system operator/technician may provide a control signal to an actuator of valve 210 to actuate valve 210 (e.g., in response to determining a safety concern, to receive a sample, etc.).

In some embodiments, valve 210 is configured to obtain portions of fluid from pipeline 208 for sampling purposes. This may be performed automatically, manually, or a combination thereof as described above. The sample(s) may be provided to analytics 212 for processing, as described in detail above. Valve 210 is shown to include plug 302, cap 304, locking nut 306, valve spring 308, spring support 310, bonnet 312, body bonnet seal 314, quad seal 316, retainer 318, stem 320, seat retainer 322, O-ring 324, seat 326, body 328, outlet connection 330, and inlet connection 332.

In a general embodiment, valve 210 is configured to receive fluid from the pipeline 208 at an inlet port within inlet connection 332 and provide fluid to an outlet port within outlet connection 330. In some embodiments, this fluid flow is performed to maintain pressure within system 200. For example, valve 210 is fluidly coupled to pipeline 208, but is closed. As pressure builds within pipeline 208, the pressure causes valve spring 308 to contract, thus opening a path between the inlet port within inlet connection 332 the outlet port within outlet connection 330. Once the pressure returns to an acceptable level, valve spring 308 may release and close the path. Of course, the pressure that engages the valve to provide/restrict fluid flow may be based on properties of valve 210 (e.g., spring constant of valve spring 308, etc.) and as such, the implementation of one or more valve(s) 210 can be performed such that relief in the system is provided at appropriate levels of pressure within system 200.

Referring now to FIGS. 3B-C, a zoomed-in cross-sectional diagram of valve 210 is shown, according to some embodiments. FIGS. 3B-C show a detailed diagram of stem 320 and seat 326 of valve 210. In some embodiments, when valve 210 is engaged to open a fluid path this is in part due to a top surface of seat 326 coming into contact with a bottom surface of stem 320. As a result, the consistent contact between these two components of valve 210 can degrade the operability of stem 320, seat 326, or a combination of both, resulting in one or more operation issues (e.g., fluid leakage, inoperability, pressure leakage, erosion, etc.).

While it may not be directly shown in FIGS. 3A-C, in some embodiments, seat 326 may include a layer of material (e.g., a substrate) disposed, placed, coupled, and/or coated on the top surface of seat 326 that comes into contact with bottom surface of stem 320. In other embodiments, stem 320 may include a layer of material disposed, placed, coupled, and/or coated on the bottom surface of stem 320 that comes into contact with top surface of seat 326. In other embodiments, both seat 326 may include a layer of material disposed, placed, coupled, and/or coated on the top surface of seat 326 that comes into contact with bottom surface of stem 320 and stem 320 may include a layer of material disposed, placed, coupled, and/or coated on the bottom surface of stem 320 that comes into contact with top surface of seat 326.

In any of the above described embodiments, the layer of material is disposed substantially between the stem 320 and the seat 236. The layer of material may be or include polycrystalline diamond (PCD) that, when applied to the surfaces of at least one of the seat 326 or stem 320, improves the durability during contact and reduces erosion. That is, the layer of material advantageously mitigates against erosion of at least one of the stem 320 or the seat 326. In some embodiments, seat 326 and/or stem 320 are manufactured out of PCD (e.g., at least in part, entirely, etc.) and no layer of PCD material needs to be coated on.

As briefly described above, the engagement components of a sampling valve (e.g., valve 210, valve 211), such as the stem 320 and the seat 236, may be manufactured at least in part out of carbine, high-content cobalt, high-content cobalt bonded with tungsten, or any combination thereof. For example, the engagement components of the sampling valve may be manufactured out of tungsten and alloys thereof In some embodiments, the engagement components are manufactured from a Tungsten Cobalt alloy of between 5 percent and 40 percent Cobalt with respect to Tungsten by atomic weight (e.g., 20 percent Cobalt and 80 percent Tungsten by atomic weight). In some embodiments, the alloy includes tertiary metals such as titanium, tantalum, etc. In some embodiments, the material for the engagement component is more malleable than pure Tungsten to prevent cracking due to the interface with the polycrystalline diamond (PCD) material. The material used to manufacture the stem 320 and/or the seat 236 advantageously mitigates against erosion, flaking, and/or cracking.

In any of the above described embodiments, the improved erosion resistance of the sampling valve (e.g., valve 210, valve 211) enables the sampling valve (e.g., valve 210, valve 211) to be used in conditions which may cause erosion. For example, the fluid sampled by the sampling valve (e.g., valve 210, valve 211) may include a higher amount of sediment or other contaminants. For example, hydrocarbons obtained from particular geographic locations may include more sediment that other geographic locations. For example, hydrocarbons obtained from some portions of North America, such as Canada, may include more sediment compared to hydrocarbons obtained from other portions of North America, such as the Southern United States. Further, hydrocarbons obtained from India may include wax which may also increase the amount sediments. The improved erosion resistance of the sampling valve (e.g., valve 210, valve 211) enables the sampling valve (e.g., valve 210, valve 211) to mitigate against additional erosion caused by sediments or other contaminants in the sampled fluids.

Furthermore, the improved erosion resistance of the sampling valve (e.g., valve 210, valve 211) enables the sampling valve (e.g., valve 210, valve 211) to be used in conditions where erosion to the sampling valve (e.g., valve 210, valve 211) may lead to the sampled fluid leaking out of the sampling valve and cause damage to surrounding equipment and/or to the environment. For example, in underwater hydrocarbon drilling, it may be desirable to sample the drilled hydrocarbon without leaking hydrocarbon into the surrounding water. The improved erosion resistance of the sampling valve (e.g., valve 210, valve 211) enables the sampling valve (e.g., valve 210, valve 211) to mitigate against erosion and eventual leaking of the sampled fluid.

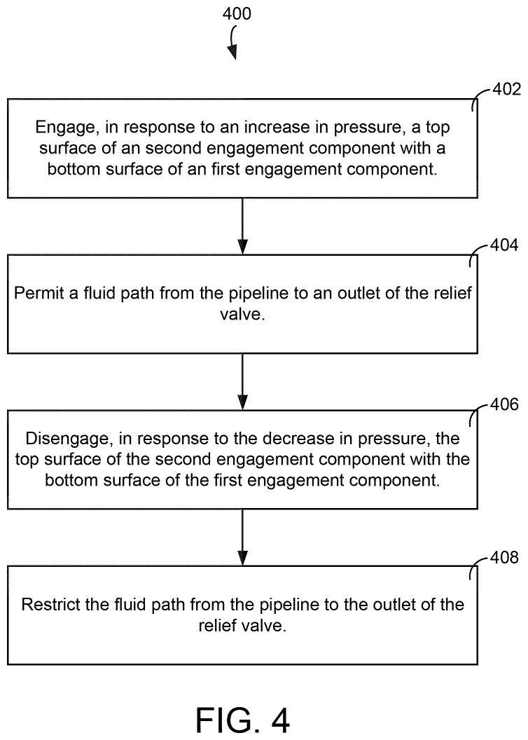

Referring now to FIG. 4, a process 400 for permitting fluid from an inlet of a valve to an outlet of a valve is shown, according to some embodiments. Process 400 may be performed by valve 210, an actuator coupled to valve 210, analytics 212, or any combination thereof

Process 400 is shown to include engaging, in response to an increase in pressure, a top surface of a second engagement component with a bottom surface of an first engagement component (step 402) and permitting a fluid path from the pipeline to an outlet of the relief valve (step 404). In some embodiments, the top surface of the second engagement component may refer to the top surface of seat 326 and the bottom surface of the first engagement component may refer to the bottom surface of stem 320.

Process 400 is shown to include disengaging, in response to the decrease in pressure, the top surface of the second engagement component with the bottom surface of the first engagement component (step 406) restricting the fluid path from the pipeline to the outlet of the relief valve (step 408). In some embodiments, these steps may be part of an operation cycle that is performed hundreds or even thousands times per day. The engagement between the first engagement component and the second engagement component, therefore, can occur frequently that leads to erosion and degradation of valve 210.

### Configuration of Exemplary Embodiments

As utilized herein, the terms “approximately,” “about,” “substantially”, and similar terms are intended to have a broad meaning in harmony with the common and accepted usage by those of ordinary skill in the art to which the subject matter of this disclosure pertains . It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the disclosure as recited in the appended claims.

It should be noted that the term “exemplary” and variations thereof, as used herein to describe various embodiments, are intended to indicate that such embodiments are possible examples, representations, or illustrations of possible embodiments (and such terms are not intended to connote that such embodiments are necessarily extraordinary or superlative examples).

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

The term “or,” as used herein, is used in its inclusive sense (and not in its exclusive sense) so that when used to connect a list of elements, the term “or” means one, some, or all of the elements in the list. Conjunctive language such as the phrase “at least one of X, Y, and Z,” unless specifically stated otherwise, is understood to convey that an element may be either X, Y, Z; X and Y; X and Z; Y and Z; or X, Y, and Z (i.e., any combination of X, Y, and Z). Thus, such conjunctive language is not generally intended to imply that certain embodiments require at least one of X, at least one of Y, and at least one of Z to each be present, unless otherwise indicated.

References herein to the positions of elements (e.g., “top,” “bottom,” “above,” “below”) are merely used to describe the orientation of various elements in the FIGURES. It should be noted that the orientation of various elements may differ according to other exemplary embodiments, and that such variations are intended to be encompassed by the present disclosure.

The hardware and data processing components used to implement the various processes, operations, illustrative logics, logical blocks, modules and circuits described in connection with the embodiments disclosed herein may be implemented or performed with a general purpose single- or multi-chip processor, a digital signal processor (DSP), an application specific integrated circuit (ASIC), a field programmable gate array (FPGA), or other programmable logic device, discrete gate or transistor logic, discrete hardware components, or any combination thereof designed to perform the functions described herein. A general purpose processor may be a microprocessor, or, any conventional processor, controller, microcontroller, or state machine. A processor also may be implemented as a combination of computing devices, such as a combination of a DSP and a microprocessor, a plurality of microprocessors, one or more microprocessors in conjunction with a DSP core, or any other such configuration. In some embodiments, particular processes and methods may be performed by circuitry that is specific to a given function. The memory (e.g., memory, memory unit, storage device) may include one or more devices (e.g., RAM, ROM, Flash memory, hard disk storage) for storing data and/or computer code for completing or facilitating the various processes, layers and modules described in the present disclosure. The memory may be or include volatile memory or non-volatile memory, and may include database components, object code components, script components, or any other type of information structure for supporting the various activities and information structures described in the present disclosure. According to an exemplary embodiment, the memory is communicably connected to the processor via a processing circuit and includes computer code for executing (e.g., by the processing circuit or the processor) the one or more processes described herein.

The present disclosure contemplates methods, systems and program products on any machine-readable media for accomplishing various operations. The embodiments of the present disclosure may be implemented using existing computer processors, or by a special purpose computer processor for an appropriate system, incorporated for this or another purpose, or by a hardwired system. Embodiments within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. Combinations of the above are also included within the scope of machine-readable media. Machine-executable instructions include, for example, instructions and data which cause a general purpose computer, special purpose computer, or special purpose processing machines to perform a certain function or group of functions.

Although the figures and description may illustrate a specific order of method steps, the order of such steps may differ from what is depicted and described, unless specified differently above. Also, two or more steps may be performed concurrently or with partial concurrence, unless specified differently above. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

It is important to note that the construction and arrangement of the apparatus as shown in the various exemplary embodiments is illustrative only. Additionally, any element disclosed in one embodiment may be incorporated or utilized with any other embodiment disclosed herein. Although only one example of an element from one embodiment that can be incorporated or utilized in another embodiment has been described above, it should be appreciated that other elements of the various embodiments may be incorporated or utilized with any of the other embodiments disclosed herein.

What is claimed is:

1. A relief valve comprising:
a first engagement component coupled with a valve spring; and
a second engagement component axially aligned with the first engagement component and configured to engage with the first engagement component during an operation cycle to translate the first engagement component along an axis in response to a pressure of a fluid within the relief valve being above a predetermined threshold; and
a carbide substrate disposed between the first engagement component and the second engagement component when the second engagement component engages the first engagement component.

2. The relief valve of claim 1, wherein the carbide substrate is fused with diamond particles to form polycrystalline diamond (PCD).

3. The relief valve of claim 1, wherein the operation cycle comprises:
engaging, in response to an increase in the pressure, a top surface of the second engagement component with a bottom surface of the first engagement component;
permitting a fluid path from a pipeline to an outlet of the relief valve;
disengaging, in response to a decrease in the pressure, the top surface of the second engagement component with the bottom surface of the first engagement component; and
restricting the fluid path from the pipeline to the outlet of the relief valve.

4. The relief valve of claim 3, wherein:
the first engagement component is a valve stem of the relief valve;
the second engagement component is a valve seat of the relief valve;
the pipeline is an unrefined oil pipeline configured to provide the fluid to a refining process, and
the fluid is crude oil.

5. The relief valve of claim 4, wherein the operation cycle of the relief valve is performed more than one thousand times per day.

6. The relief valve of claim 1, wherein the relief valve is a sampling relief valve configured to obtain a sample of the fluid flowing through a pipeline.

7. The relief valve of claim 6, wherein the relief valve is fluidly coupled with an analytics system, the analytics system configured to:
receive the sample from the relief valve in response to a completion of the operation cycle; and
analyze one or more properties of the sample to determine an amount of sediment within the sample.

8. The relief valve of claim 1, wherein at least one of the first engagement component or the second engagement component is manufactured from a Tungsten Cobalt alloy of between 5 percent and 40 percent Cobalt with respect to Tungsten by atomic weight.

9. A relief valve in a hydrocarbon site, the relief comprising:
an inlet port configured to receive a fluid from a pipeline;
a first engagement component;
a second engagement component configured to engage with the first engagement component during an operation cycle such that the second engagement component causes the first engagement component to translate the stem along an axis in response to a pressure of the fluid within the relief valve being above a predetermined threshold; and
a carbide substrate disposed between the first engagement component and the second engagement component.

10. The relief valve of claim 9, wherein the carbide substrate is fused with diamond particles to form polycrystalline diamond (PCD).

11. The relief valve of claim 9, wherein the operation cycle comprises:
engaging, in response to an increase in the pressure, a top surface of the second engagement component with a bottom surface of the first engagement component;
permitting a fluid path from the pipeline to an outlet of the relief valve;
disengaging, in response to the decrease in the pressure, the top surface of the second engagement component with the bottom surface of the first engagement component; and
restricting the fluid path from the pipeline to the outlet of the relief valve.

12. The relief valve of claim 11, wherein:
the first engagement component is a valve stem of the relief valve;
the second engagement component is a valve seat of the relief valve;
the pipeline is an unrefined oil pipeline configured to provide the fluid to a refining process, and
the fluid is crude oil.

13. The relief valve of claim 11, wherein the operation cycle of the relief valve is performed more than one thousand times per day.

14. The relief valve of claim 9, wherein the relief valve is a sampling relief valve configured to obtain a sample of the fluid flowing through the pipeline.

15. The relief valve of claim 14, wherein the relief valve is fluidly coupled with an analytics system, the analytics system configured to:
receive the sample from the relief valve in response to a completion of the operation cycle; and
analyze one or more properties of the sample to determine an amount of sediment within the sample.

16. A valve comprising:
a first engagement component; and
a second engagement component configured to engage with the first engagement component; and
a carbide substrate disposed between the first engagement component and the second engagement component such that the carbide substrate contacts the first engagement component and the second engagement component when the second engagement component engages the first engagement component.

17. The valve of claim 16, wherein the carbide substrate is fused with diamond particles to form polycrystalline diamond (PCD).

18. The valve of claim 16, wherein the second engagement component is configured to engage with the first engagement component during an operation cycle, wherein the operation cycle comprises:
engaging, in response to an increase in a pressure of a fluid within the valve, a top surface of the second engagement component with a bottom surface of the first engagement component;
permitting a fluid path from an inlet of the valve to an outlet of the valve;
disengaging, in response to a decrease in the pressure of the fluid within the valve, the top surface of the second engagement component with the bottom surface of the first engagement component; and
restricting the fluid path from the inlet of the valve to the outlet of the relief valve.

19. The valve of claim 17, wherein:
the first engagement component is a valve stem of the relief valve;
the second engagement component is a valve seat of the relief valve;
the inlet is configured to receive the fluid from an unrefined oil pipeline configured to provide the fluid to a refining process, and
the fluid is crude oil.

20. The valve of claim 17, wherein the valve is fluidly coupled with an analytics system, the analytics system configured to:
receive the fluid from the relief valve in response to a completion of the operation cycle; and
analyze one or more properties of the fluid to determine an amount of sediment within the fluid.
